(19) United States
(12) Patent Application Publication
(10) Pub. No.: US 2016/0243182 A1
(43) Pub. Date: Aug. 25, 2016

(54) USE OF CISSAMPELOS PAREIRA EXTRACTS FOR TREATING DENGUE

(71) Applicants: SUN PHARMACEUTICAL INDUSTRIES LIMITED, Mumbai (IN); INTERNATIONAL CENTRE FOR GENETIC ENGINEERING AND BIOTECHNOLOGY, New Delhi (IN); DEPARTMENT OF BIOTECHNOLOGY, New Delhi (IN)

(72) Inventors: Pradip Kumar BHATNAGAR, Exton, PA (US); Chandra Kant KATIYAR, Gurgaon (IN); Navin KHANNA, New Delhi (IN); Poornima TYAGI, Ghaziabad (IN); Dilip Jatashankar UPADHYAY, Kalyan (IN); Sathyamangalam SWAMINATHAN, Hyderabad (IN); Kona SRINIVAS, East Godavari (IN); Navin Kumar SHARMA, Northwest Delhi (IN); Anil KANAUJIA, Kanpur (IN); Ruchi SOOD, New Delhi (IN); Tarani Kanta BARMAN, Nalbari (IN); Smita SINGHAL, Gautam Buddh Nagar (IN); Gyanesh SHUKLA, Jalaun (IN); Rajeev DUGGAR, Jodhpur (IN); Pawan Kumar PAREEK, Bikaner (IN); Yatendra Kumar GUPTA, New Delhi (IN); Yogendra SINGH, Gurgaon (IN); Seema KHAN, New Delhi (IN); Rajendra RAUT, New Delhi (IN); Raj Kumar SHIRUMALLA, New Delhi (IN); Vijay KANOJE, Pune (IN); Ramesh SUBBARAYAN, Bangalore (IN); Ravisankar RAJERETHINAM, Clementi (SG)

(21) Appl. No.: 15/077,238

(22) Filed: Mar. 22, 2016

Related U.S. Application Data
(63) Continuation-in-part of application No. 13/145,732, filed on Jan. 10, 2012.

(30) Foreign Application Priority Data
Jan. 23, 2009 (IN) 141/DEL/2009
Dec. 17, 2015 (IN) 11520/DELNP/2015

(51) Int. Cl. A61K 36/59; A61K 31/167

(52) U.S. Cl. CPC A61K 36/59; A61K 31/167

(57) ABSTRACT

The present invention relates to an extract of Cissampelos pareira, its pharmaceutical compositions, and its therapeutic use in the prevention and treatment of dengue. It also relates to processes for the preparation of these extracts.

### FIELD OF THE INVENTION

The present invention relates to an extract of Cissampelos pareira, its pharmaceutical compositions, and its therapeutic use in the prevention and treatment of dengue. It also relates to processes for the preparation of these extracts.

### BACKGROUND OF THE INVENTION

Dengue disease remains a major public health concern around the world. The incidence of dengue has grown dramatically around the world in recent decades. Dengue occurs in tropical and sub-tropical climates worldwide, mostly in urban and semi-urban areas. Severe dengue is a leading cause of serious illness and death among children in many Asian and Latin American countries. According to World Health Organization (WHO) estimates, ˜2.5 billion people around the globe are at risk of dengue, with ˜50 million infections worldwide annually. Dengue is spread to humans through Aedes mosquitoes, which serve as carriers of the disease-causing viruses. There are four serotypes of dengue viruses (DENV-1, -2, -3, and -4) belonging to the family Flaviviridae. Recovery from infection by one serotype of dengue virus provides lifelong immunity against that particular serotype. However, cross-immunity to the other serotypes after recovery is only partial and temporary. Subsequent infections by other serotypes increase the risk of developing severe dengue. Infection with DENVs may be asymptomatic, or may result in a range of clinical symptoms from mild dengue fever (DF) to severe and potentially fatal dengue haemorrhagic fever (DHF) and dengue shock syndrome (DSS). The clinical symptoms for mild dengue fever include high fever, severe headache, pain behind the eyes, muscle and joint pains, nausea, vomiting, swollen glands, and rash. Symptoms usually last for 2-7 days, after an incubation period of 4-10 days following the bite from an infected mosquito. The clinical symptoms for severe dengue appear due to plasma leaking, fluid accumulation, respiratory distress, severe bleeding, or organ impairment. Despite the alarming impact on both human health and the global economy, there is no specific treatment of dengue yet available. Though some live attenuated dengue vaccines are being developed, the challenges faced in dengue vaccine development remain high.

Thus, there exists an urgent need for an effective dengue treatment that can shorten the duration of the illness, reduce the severity of common symptoms, prevent the development of severe complications, and is easy to formulate. Furthermore, it is highly desirable to develop a dengue treatment that can reduce the viral load at an early stage, such that it may potentially prevent dengue fever as well as a life-threatening severe form of dengue.

The present invention fulfills this unmet need by providing an effective, patient-compliant dengue treatment. Cissampelos pareira extracts help to effectively prevent, and treat, the dengue viral disease. The present inventors have found that the extracts of Cissampelos pareira Linn (Cipa extract) are potent inhibitors of all four DENVs in cell-based assays, assessed in terms of viral NS1 antigen secretion using ELISA, as well as viral replication, based on plaque assays. Virus yield reduction assays showed that the extracts of Cissampelos pareira decrease viral titers by an order of magnitude. The extracts of Cissampelos pareira conferred statistically significant protection against DENV infection using the AG129 mouse model. Surprisingly, it been discovered that the potency of Cissampelos pareira extracts extend over a wide range of viral loads, including the initial stage viral load, which could subsequently prevent the life-threatening severe form of dengue. Further, the present inventors have determined that both Cissampelos pareira extracts and paracetamol show a synergistic effect in decreasing the body temperature. Also, the dengue disease predisposes some patients to hemorrhagic manifestations and tends to be associated with lowered platelet counts. Therefore, it also becomes important to assess if Cissampelos pareira extracts have any undesired effect on erythrocytes and platelets. The present inventors have determined that Cissampelos pareira extracts did not have any discernible effect on platelet counts or on erythrocyte viability. They have also determined that the extracts also possessed the ability to downregulate the secretion of pro-inflammatory cytokines, particularly TNF-α and IL-1β. Further, extracts of Cissampelos pareira showed no evidence of toxicity.

### SUMMARY OF THE INVENTION

The present invention provides an extract of Cissampelos pareira, its pharmaceutical compositions, and its therapeutic use in the prevention and treatment of dengue. It also relates to processes for the preparation of these extracts. It further provides the activity of these extracts against dengue virus in mammals. Further, it provides the synergistic antipyretic effect of Cissampelos pareira extract in combination with paracetamol. It also provides the anti-inflammatory effect of Cissampelos pareira extracts with no adverse effect on platelet counts and on erythrocyte viability. Further, these extracts did not show any toxic effects.

### BRIEF DESCRIPTION OF THE DRAWINGS

FIG. 1: Schematic representation of the antiviral screening assays.

FIG. 2: Inhibition of DENV antigen and virus production by the treatment of methanolic extract.

FIG. 3: Effect of pre-incubation time on antiviral activity of methanolic extract.

FIG. 4: Evaluation of in-vivo protective efficacy of methanolic extract.

FIG. 5: Analysis of interaction between paracetamol and methanolic extract.

FIG. 6: Effect of methanolic extract on platelets.

FIG. 7: Effect of methanolic extract on RBCs.

### DETAILED DESCRIPTION OF THE INVENTION

A first aspect of the present invention provides an extract of Cissampelos pareira for use in the treatment of dengue virus infection in mammals.

A second aspect of the present invention provides a pharmaceutical composition for use in the treatment of dengue virus infection in mammals comprising an extract of Cissampelos pareira and one or more pharmaceutically acceptable excipients.

A third aspect of the present invention provides a pharmaceutical composition for use in the treatment of dengue virus infection in mammals, comprising:

(a) an extract of Cissampelos pareira; and

(b) paracetamol

wherein (a) and (b) are administered together as a single pharmaceutical composition or are co-administered simultaneously or sequentially.

A fourth aspect of the present invention provides a method of treating dengue virus infection in mammals comprising administering a pharmaceutical composition comprising:

(a) an extract of Cissampelos pareira; and

(b) paracetamol

wherein (a) and (b) are administered together as a single pharmaceutical composition or are co-administered simultaneously or sequentially.

A fifth aspect of the present invention provides an extract of Cissampelos pareira to reduce the viral load at an early stage in the treatment of dengue virus infection in mammals.

A sixth aspect of the present invention provides a method of reducing the viral load at an early stage in the treatment of dengue virus infection in mammals comprising administering an extract of Cissampelos pareira.

A seventh aspect of the present invention provides an extract of Cissampelos pareira for use in the treatment of dengue virus infection in mammals, wherein the extract exhibits a platelet protective effect.

An eighth aspect of the present invention provides a method of treating dengue virus infection in mammals comprising administering an extract of Cissampelos pareira, wherein the extract exhibits a platelet protective effect.

A ninth aspect of the present invention provides an extract of Cissampelos pareira for use in the treatment of dengue virus infection in mammals, wherein the extract exhibits an erythrocyte protective effect.

A tenth aspect of the present invention provides a method of treating dengue infection in mammals comprising administering an extract of Cissampelos pareira, wherein the extract exhibits an erythrocyte protective effect.

An eleventh aspect of the present invention provides an extract of Cissampelos pareira to reduce the viral load at an early stage in the treatment of dengue virus infection in mammals, wherein the extract exhibits a platelet protective effect.

A twelfth aspect of the present invention provides a method for reducing the viral load at an early stage in the treatment of dengue virus infection in mammals comprising administering an extract of Cissampelos pareira, wherein the extract exhibits a platelet protective effect.

A thirteenth aspect of the present invention provides an extract of Cissampelos pareira to reduce the viral load at an early stage in the treatment of dengue virus infection in mammals, wherein the extract exhibits an erythrocyte protective effect.

A fourteenth aspect of the present invention provides a method for reducing the viral load at an early stage in the treatment of dengue virus infection in mammals comprising administering an extract of Cissampelos pareira, wherein the extract exhibits an erythrocyte protective effect.

A fifteenth aspect of the present invention provides a pharmaceutical composition comprising an extract of Cissampelos pareira and one or more pharmaceutically acceptable excipients to reduce the viral load at an early stage in the treatment of dengue virus infection in mammals, wherein the extract exhibits a platelet protective effect.

A sixteenth aspect of the present invention provides a pharmaceutical composition comprising an extract of Cissampelos pareira and one or more pharmaceutically acceptable excipients to reduce the viral load at an early stage in the treatment of dengue virus infection in mammals, wherein the extract exhibits an erythrocyte protective effect.

In one embodiment of the above aspects, the extract is an alcoholic extract, a hydroalcoholic extract, or an aqueous extract. In a preferred embodiment, the extract is an alcoholic extract. In a more preferred embodiment, the extract is a methanolic extract. The methanol in the methanolic extract may be removed completely by evaporation to obtain a dried extract. The dried extract may be lyophilized to form a powder, which can then be filled into a capsule of suitable size.

In another embodiment of the above aspects, the extract of Cissampelos pareira is used for the prevention of dengue virus infection.

Cissampelos pareira belongs to a family Menispermaceae and is a climbing shrub distributed throughout the warm parts of Asia, East Africa, and North and South America, and is common in India and Sri Lanka. It is also commonly known as Velvet Leaf or Patha or Ambasthaki. It is common in warm and dry regions of tropical and sub-tropical parts of India up to an altitude of about 1500 meters. It is found in Himachal Pradesh, Chota Nagpur, Bihar, West Bengal, Punjab, Rajasthan particularly in the east of Aravalli, the hilly forests of Marathwada, Konkan, Deccan, the Bababuden hills of Mysore, and Tamil Nadu (Ayurvedic Pharmacopoeia of India, First Edition, Part 1, Vol 1, p. 92-93, Govt. of India, Ministry of Health and Family Welfare, Dept. of Indian System of Medicine and Homeopathy, New Delhi; The Wealth of India, A Dictionary of Indian Raw Materials and Industrial Products, Raw Materials, Vol II, Council of Scientific and Industrial Research, Delhi; Database on Medicinal Plants Used in Ayurveda, Vol 2, Central Council for Research in Ayurveda and Siddha, Dept. of Indian system of Medicine and Homeopathy, New Delhi).

Paracetamol chemically is N-(4-hydroxyphenyl) acetamide. It is also commonly known as acetaminophen. It is a well-known antipyretic which has been used for a number of years. The present invention provides a synergistic antipyretic effect of paracetamol with the extract of Cissampelos pareira. The present invention incorporates the safe and effective use of paracetamol in combination with the extracts of Cissampelos pareira for treating or preventing dengue viral infections. Paracetamol and the extracts of Cissampelos pareira can be administered together as a single pharmaceutical composition or can be co-administered simultaneously or sequentially.

The term “alcoholic extract,” as used herein, includes any alcohol-based extract, for example, methanolic, ethanolic, n-propanolic, isopropanolic, n-butanolic, iso-butanolic or t-butanolic extract of Cissampelos pareira. In particular, the alcoholic extract is a methanolic extract.

The term “hydroalcoholic extract,” as used herein, includes an extract prepared by using a mixture of alcohol and purified water. Examples of alcohols are methanol, ethanol, n-propanol, isopropanol, n-butanol, iso-butanol, and t-butanol. In particular, a 1:1 mixture of alcohol and purified water is used.

The term “aqueous extract,” as used herein, includes a purified water extract of Cissampelos pareira.

The extracts of Cissampelos pareira are prepared by extracting the plant mass of Cissampelos pareira with one or more solvents selected from methanol, ethanol, n-propanol, isopropanol, n-butanol, iso-butanol, t-butanol, purified water, and mixtures thereof, concentrating the extract, and drying the extract.

The term “plant mass of Cissampelos pareira,” as used herein, refers to the whole plant, which includes aerial parts, for example, fruits, flowers, leaves, branches, stem bark, stems, seeds or heartwood, and roots.

The term “minimum lethal dose (MLD),” as used herein, refers to the dose that can cause clinical symptoms and 90% to 100% death 3-4 weeks post-challenge.

The term “pharmaceutical composition,” as used herein, includes any composition that can effectively deliver the extracts of Cissampelos pareira to the desired site of action to treat or prevent dengue viral infection. The composition can be delivered by any suitable route of administration, such as oral, nasal, pulmonary, transdermal, or rectal. The pharmaceutical composition includes one or more pharmaceutically acceptable excipients. The oral pharmaceutical composition can be in the form of powder, pellets, granules, spheroids, mini-tablets, caplets, tablets, or capsules. The powder can be in the form of a lyophilized powder filled, with pharmaceutically acceptable excipients, into a capsule of suitable size.

The term “pharmaceutically acceptable excipients,” as used herein, includes diluents, binders, disintegrants, lubricants, glidants, polymers, flavoring agents, surfactants, preservatives, antioxidants, buffers, and tonicity modifying agents.

Examples of diluents include microcrystalline cellulose, powdered cellulose, starch, starch pregelatinized, dextrates, lactitol, fructose, sugar compressible, sugar confectioners, dextrose, lactose, calcium phosphate-dibasic, calcium phosphate-tribasic, calcium sulfate, and mixtures thereof.

Examples of binders include a water-soluble starch, for example, pregelatinized starch; a polysaccharide, for example, agar, gum acacia, dextrin, sodium alginate, tragacanth gum, xanthan gum, hyaluronic acid, pectin, or sodium chondroitin sulfate; a synthetic polymer, for example, polyvinylpyrrolidone, polyvinyl alcohol, carboxyvinyl polymer, polyacrylic acid-series polymer, polylactic acid, or polyethylene glycol; a cellulose ether, for example, methyl cellulose, ethyl cellulose, carboxymethyl cellulose, hydroxyethyl cellulose, hydroxypropyl cellulose, or hydroxypropyl methyl cellulose; and mixtures thereof.

Examples of disintegrants include calcium carbonate, carboxymethyl cellulose or a salt thereof, for example, croscarmellose sodium, crosslinked povidone, low-substituted hydroxypropyl cellulose, and sodium starch glycolate.

Examples of lubricants/glidants include talc, magnesium stearate, hydrogenated vegetable oils, sodium stearyl fumarate, calcium stearate, colloidal silicon dioxide, Aerosil®, stearic acid, sodium lauryl sulphate, sodium benzoate, polyethylene glycol, hydrogenated castor oil, sucrose esters of fatty acids, microcrystalline wax, yellow beeswax, white beeswax, and mixtures thereof.

Examples of flavoring agents include synthetic flavor oils and flavoring aromatics; natural oils or extracts from plants, leaves, flowers, and fruits; and combinations thereof. These may include cinnamon oil, oil of wintergreen, peppermint oils, bay oil, anise oil, eucalyptus, thyme oil, vanilla, citrus oil, including lemon, orange, lime, and grapefruit, and fruit essences including apple, banana, grape, pear, peach, strawberry, raspberry, cherry, plum, pineapple, and apricot.

Examples of surfactants include anionic surfactants, for example, a sulfonic acid or a salt thereof such as benzenesulfonic acid, dodecylbenzenesulfonic acid, or dodecanesulfonic acid; an alkyl sulfate, for example, sodium dodecyl sulfate or sodium lauryl sulfate; cationic surfactants, for example, a tetraalkylammonium salt such as a tetraalkylammonium halide, benzethonium chloride, benzalkonium chloride, or cetylpyridinium chloride; a nonionic surfactant, for example, a (poly) oxyethylene sorbitan long-chain fatty acid ester such as a polyoxyethylene sorbitan monolaurate, for example, a polysorbate; amphoteric surfactants, for example, a glycin compound such as dodecyl-di-(aminoethyl)glycin, a betaine compound such as betaine or dimethyldodecylcarboxybetaine, and a phosphatidic acid derivative such as lecithin; polymeric surfactants, for example, a polyoxyethylene polyoxypropylene glycol such as Pluronic® or poloxamer; and mixtures thereof.

Examples of buffers include phosphate buffers such as dihydrogen sodium phosphate, citrate buffers such as sodium citrate, meglumine, tri(hydroxymethyl) aminomethane, and mixtures thereof.

Examples of tonicity modifying agents include sodium chloride, mannitol, dextrose, glucose, lactose, sucrose, and mixtures thereof.

Examples of solvents for the preparation of the pharmaceutical composition include water; water miscible organic solvents, for example, isopropyl alcohol or ethanol; dipolar aprotic solvents; methylene chloride; acetone; polyethylene glycol; polyethylene glycol ether; polyethylene glycol derivatives of a mono- or di-glyceride; buffers; organic solvents; and combinations thereof.

While the following examples are provided to certain embodiments of the invention, these are not intended to be limiting to the scope of the invention.

### EXAMPLES

### Example 1

### Preparation of a Methanolic Extract of Cissampelos Pareira

Pulverized Cissampelos pareira aerial parts (100 kg) were charged into an extractor. Methanol (500 liter) was added, and the extraction was performed at a temperature ranging from room temperature to the boiling point of the solvent for about 16 hours. The extract was filtered, and then stored in a container. The extraction and filtration steps were repeated with 300 liters of methanol, twice. The filtered extracts were stored in containers. The three methanolic extracts were combined and concentrated to the maximum possible extent under reduced pressure at a low temperature. The extract was decanted into stainless steel trays, and then dried in a high vacuum oven at room temperature for about 16 hours to 18 hours.

Yield=6% to 15% w/w

The dried extract was lyophilized to form a powder. This powder was then filled into a capsule of suitable size.

### Example 2

### Preparation of a Hydroalcoholic (1:1 Methanol:Purified Water) Extract of Cissampelos Pareira

Pulverized Cissampelos pareira aerial parts (100 kg) were charged into an extractor. A mixture of methanol and purified water (250 L:250 L) was added, and the extraction was performed at a temperature ranging from room temperature to the boiling point of the solvent for about 16 hours. The extract was filtered, and then stored in a container. The extraction and filtration steps were repeated with methanol:purified water (150 L:150 L), twice. The filtered extracts were stored in containers. The three hydroalcoholic extracts were combined and concentrated to the maximum possible extent under reduced pressure at a low temperature. The extract was decanted into stainless steel trays, and then dried in a high vacuum oven at room temperature for about 16 hours to 18 hours.

Yield=10% to 25% w/w.

### Example 3

### Preparation of an Aqueous Extract of Cissampelos Pareira

Pulverized Cissampelos pareira aerial parts (100 kg) were charged into an extractor. Purified water (500 L) was added, and extraction was performed at a temperature ranging from room temperature to the boiling point of the solvent for about 16 hours. The extract was filtered, and then stored in a container. The extraction and filtration steps were repeated with 300 L of purified water, twice. The filtered extracts were stored in containers. The three aqueous extracts were combined and concentrated to maximum under reduced pressure at low temperature. The extract was decanted into stainless steel trays, and then dried in a high vacuum oven at room temperature for about 16 hours to 18 hours.

Yield=15% to 30% w/w.

### Example 4

### Biological Activity

### (a) Plaque Assay

LLCMK2 monolayers in 6 well plates were infected in duplicate with serial 10-fold dilutions (prepared in Dulbecco's Modified Eagles Medium (DMEM)+2% heat inactivated fetal calf serum (ΔFCS)) of the virus-containing samples (250 μl/well). Mock-infections were performed in parallel using an equivalent volume of virus diluent alone. Two hours later, the infected monolayers (after aspirating off the virus inoculum) were overlaid with DMEM+6% ΔFCS containing 1% methyl cellulose (2 mL/well), and then incubated for 6 days (37° C., 5% CO₂). On day 6 post-infection, the overlay was removed and the cells were fixed with a 4% formaldehyde solution (1 mL/well). The fixed cells were washed, and then stained with a 0.05% (w/v) crystal violet solution in 20% ethanol. The revealed plaques were counted to determine the virus titre, expressed as plaque-forming units (PFUs)/ml.

### (b) Cell-Based Bioassays for Antiviral Screening

(i) Type-1assay: In the initial antiviral screening assay, designated as the type-1 assay, LLCMK2 cells were seeded in 24-well plates (5×10⁵ cells/well), a day in advance. DENV-1, -2, -3, and -4 (100 PFU each) were separately pre-incubated with serial dilutions of the extracts of the present invention (corresponding to 0 μg/mL to 100 μg/mL final concentration) in 300 μl volume, at 4° C. overnight. The pre-incubation mixture was diluted with an equal volume of medium (DMEM+2% ΔFCS) and used to infect LLCMK2 cells (3 wells for each concentration at 200 μl/well) in the 24-well plate. After 2 hours of adsorption in the incubator (37° C., 5% CO₂), the infected cells were overlaid with methylcellulose-containing growth medium and processed thereafter as described for the plaque assay (a). The cells were exposed to the extracts of the present invention (in the same concentration range) in the absence of the DENV infection to assess any potential cytotoxicity. Additional control experiments were run in parallel, which included cells which were either mock-infected (negative control) or infected with DENV in the absence of the extracts of the present invention (positive control). The half-maximal inhibitory concentration (IC₅₀ value) for each extract against each DENV serotype, with reference to the positive control, which represented 100% infection (or 0% inhibition), was defined as the concentration of the extract, in μg/ml, resulting in 50% inhibition of the plaque count.

(ii) Type-2 assay: LLCMK2 cells in 24-well plates were infected with DENVs (a multiplicity of infection (MOI)=0.002) without pre-incubating with the extracts. After 2 hours of the adsorption, the virus inoculum was aspirated, the monolayer was rinsed, and then fed with the complete medium containing the extracts of the present invention (corresponding to 0 μg/mL to 200 μg/mL final concentration). After 24 hours of exposure to the extracts of the present invention, the monolayer was aspirated, and then overlaid with growth medium containing methyl cellulose and plaques developed 6 days later.

(iii) Type-3 assay: Type-3 assay was performed using Vero cells. The assay design was similar to the type-2 assay, except that following the sequential exposure of cells to DENV and the extracts of the present invention, the cells were fed with liquid growth medium instead of the methylcellulose overlay. Aliquots of the culture supernatant were withdrawn at periodic intervals up to 9 days for estimation of NS1 antigen levels (using a commercial ELISA kit) and virus titres (by plaque assay, as described in (a)).

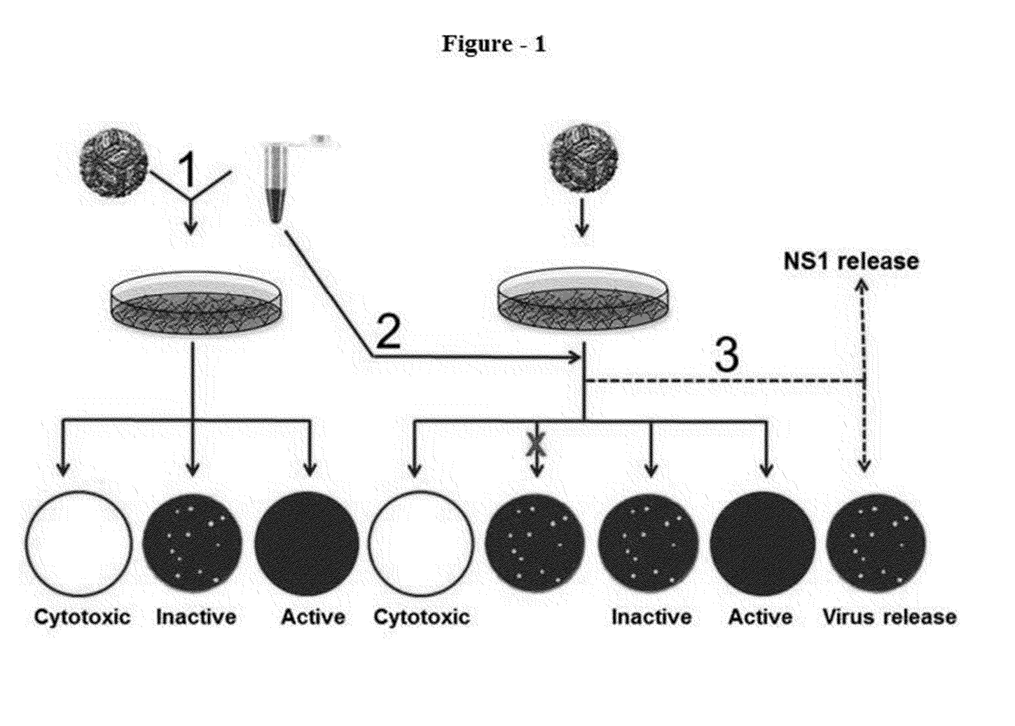

FIG. 1 provides a schematic representation of the antiviral screening assays. An outline of the three types of screening assays (indicated by numbers 1, 2 and 3) is shown. The multi-colored sphere represents DENV, and the Eppendorf tube with green liquid represents the extract of the present invention. These two were pre-incubated (1) before addition to the monolayer or added sequentially (2, 3) to the monolayer. In assays 1 and 2, the treated-monolayers were overlaid with methyl cellulose containing growth medium. Shown at the bottom are the possible outcomes of the type 1 and 2 assays. The ‘x’ mark denotes failure of entry into cells. In assay 3, liquid growth medium was added instead of the methyl cellulose overlay, followed by analysis of NS1 and virus released into the culture supernatant.

### (c) Determination of Cytotoxicity

Cytotoxicity was evaluated in two cell lines, LLCMK2 (in which the antiviral activity of the extracts were assayed) and HepG2, a commonly used liver cell surrogate for in-vitro cytotoxicity testing. Cells seeded in 96-well plates were exposed to a wide concentration range of the extracts of the invention (1 μg/mL to 200 μg/mL) for 3 days. Cell viability was assessed using a commercial MTT (3-[4,5-dimethylthiazol-2-yl]-2,5-diphenyltetrazolium bromide) assay kit, with reference to control cells that were not exposed to the extracts of the invention. The half-maximal cytotoxic concentration (CC₅₀ value) for the extract, with reference to the positive control (untreated cells) which represented 100% cell viability (or 0% cytotoxicity), was defined as the concentration of the extract, in μg/ml, resulting in 50% cytotoxicity. Selectivity index (SI) of an extract is defined as the ratio of CC₅₀ to IC₅₀ values obtained using the LLCMK2 cell line.

### (d) Inhibition of Secretion of Viral Antigen and Infectious Virus

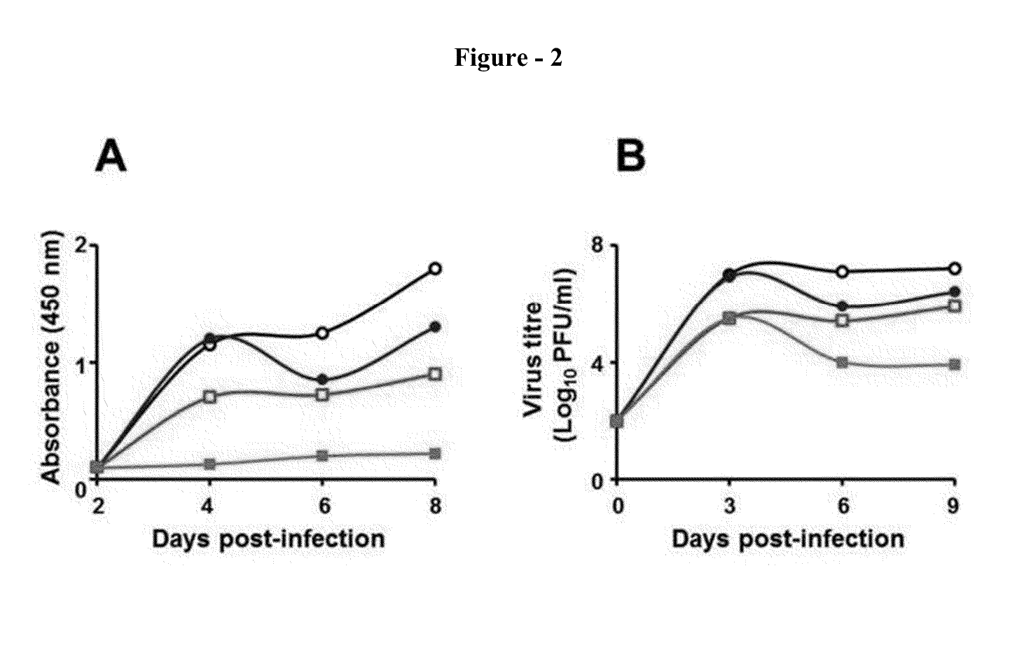

The kinetics of virus inhibition by the extracts of the present invention was analysed in a type-3 assay. Aliquots of the culture supernatant were withdrawn at regular intervals over a period of several days, and analyzed for the presence of viral NS1 antigen and infectious virus, as shown in FIG. 2. In control experiments, wherein infected cells were not exposed to the extract, NS1 antigen was detectable from day 2 onwards, and rising thereafter during the course of the experiment. In parallel experiments, the exposure of cells to the extract had a dose-dependent inhibitory effect on NS1 antigen secretion. While the inhibition resulting from exposure to a low dose of the extract was manifested after day 4 post-infection, inhibition at higher doses was evident earlier and at relatively higher magnitudes (FIG. 2A). In fact, at the highest dose of the extract tested in this experiment (100 μg/ml), the inhibition of NS1 antigen was near total for the entire duration of the experiment. The inhibition of viral antigen synthesis evident from this experiment shows that virus production would also be similarly affected. This notion was substantiated by determination of viral titers in the culture supernatants during the course of the above experiment, as shown in FIG. 2B. In the control experiment, viral titers increased steadily, reaching a plateau at day 3 post-infection. The extracts of the present invention lowered viral titers in a dose-dependent manner as seen for NS1 secretion. Thus, at the lowest concentration of the extract used, reduction in viral titers became apparent from day 4 onward. Significantly, a small increase in the extract dosage resulted in >1 log reduction in viral titer as early as day 3 post infection. At the highest dose of the extract tested (100 μg/ml), the drop in viral titers was ˜2 logs. Importantly, the reduction in viral titers was sustained over a period of several days. Surprisingly, the magnitude of inhibition appeared to be greater based on NS1 levels compared to viral titers. The data shows that the extract may have effects on NS1 antigen synthesis and release that are distinct from its effects on virus replication.

FIG. 2 depicts the kinetics of NS1 antigen (A) and infectious virus (B) released into the culture supernatant in the absence (empty black circles) and presence of the methanolic extract at 22 μg/ml (filled blue circles), 66 μg/ml (empty red squares), and 200 μg/ml (filled green squares) concentrations.

### (e) Determination of the Effect of Pre-Incubation Time and Virus Dose on the Anti-DENV Activity

To assess the influence of the duration of pre-incubation of the extracts of the present invention with DENV antiviral activity in the type-1 assay format, pre-incubation times ranging from 0 to 24 hours were tested using ˜50 PFUs of DENV-3.

To assess the effect of the size of the DENV dose on the anti-DENV efficacy of the extracts of the present invention in the pre-incubation step (at 4° C., overnight), type-1 assays were performed using DENV-3 ranging from 50 to 5000 PFUs. Each dose of DENV-3 was assayed against the extract ranging in concentration from 0 μg/mL to 200 μg/mL.

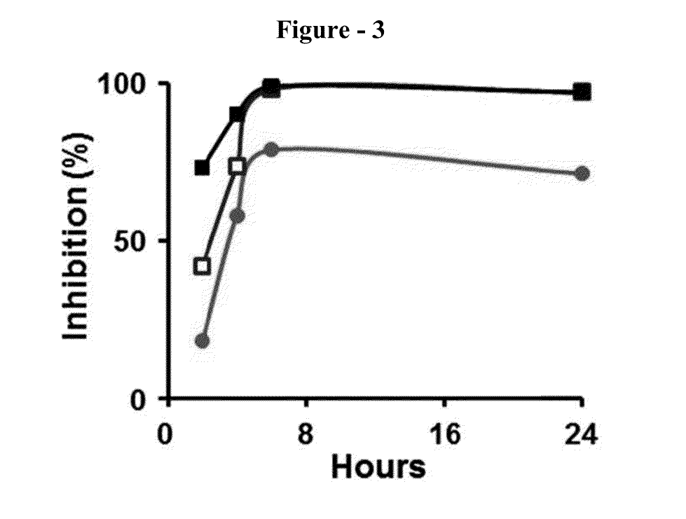

DENV-3 was pre-incubated with increasing concentrations of the extracts of the present invention for different periods of time before infection (type-1 assay) and overlay. Plaque counts obtained at the end of the experiment revealed a dose- and time-dependent virucidal effect of the extracts of the present invention on DENV-3 as depicted in FIG. 3. A converse experiment, again in type-1 format, was carried out to determine the inhibitory efficacy of the extract against DENV-3 stocks whose titers varied over 2 logs. The IC₅₀ values of methanolic extract corresponding to DENV-3 dosage of 50, 500, and 5000 PFUs were, respectively, 9.92, 12.5, and 44.45 μg/ml. This leads to the conclusion that the antiviral potency of the methanolic extract extends over a wide range of viral loads.

FIG. 3 provides the effect of pre-incubation time on the antiviral activity of the methanolic extract of the present invention. DENV-3 (50 PFU) was pre-incubated with the extract at 11 μg/ml (filled red circles), 33 μg/ml (empty blue squares), and 100 μg/ml (filled black squares) for different durations (2 to 24 hours) followed by assay of antiviral activity in a type-1 assay.

### (f) Determination of In-Vivo Protective Efficacy

To determine the in-vivo efficacy, DENV-2 (NGC) was alternately passaged between AG129 (intracranial inoculation of 10⁶ PFU) and C6/36 cells in tissue culture. After 4 to 5 such cycles of passaging, the virus was tested in AG129 mice to determine the minimum lethal dose (MLD) by i.p. injections. The challenge virus stock thus obtained was titrated, aliquotted, and stored in liquid N₂ until use. To test protective efficacy of the extracts of the present invention, AG129 mice (9 to 12 weeks old, 20 to 24 g body weight) were challenged with 10⁶ PFU (per mouse, 0.4 mL, i.p.) of the challenge DENV-2 stock.

Challenged mice were divided into groups (n=6) and treated orally with the vehicle alone (0.25% methyl cellulose) or with one of two different doses of the extracts of the present invention (at 125 mg and 250 mg/kg body weight). The methanol in the methanolic extract administered to the mice was removed completely by evaporation. The resultant methanol-free paste was thoroughly re-suspended in 0.25% methyl cellulose water and administered orally to the infected mice. The volume of the oral dose was adjusted in accordance with the body weight of each animal (10 mL/Kg/dose) and administered by a trained veterinarian using a specially designed mouse feeder needle fitted with a graduated 1 mL disposable syringe. The treatment was initiated 2 hours post-infection and continued twice daily for 5 consecutive days. Animals were monitored twice daily for a period of 35 days for clinical symptoms and mortality. A control group that was not virus-challenged, but which received the extract of the present invention (250 mg/kg), was also tested in parallel. At the end of the experiment, the survival data was used to plot Kaplan Meier survival curves and analysed by the log rank test (Mantel-Cox test) for statistical significance using GraphPad Prism 5 software.

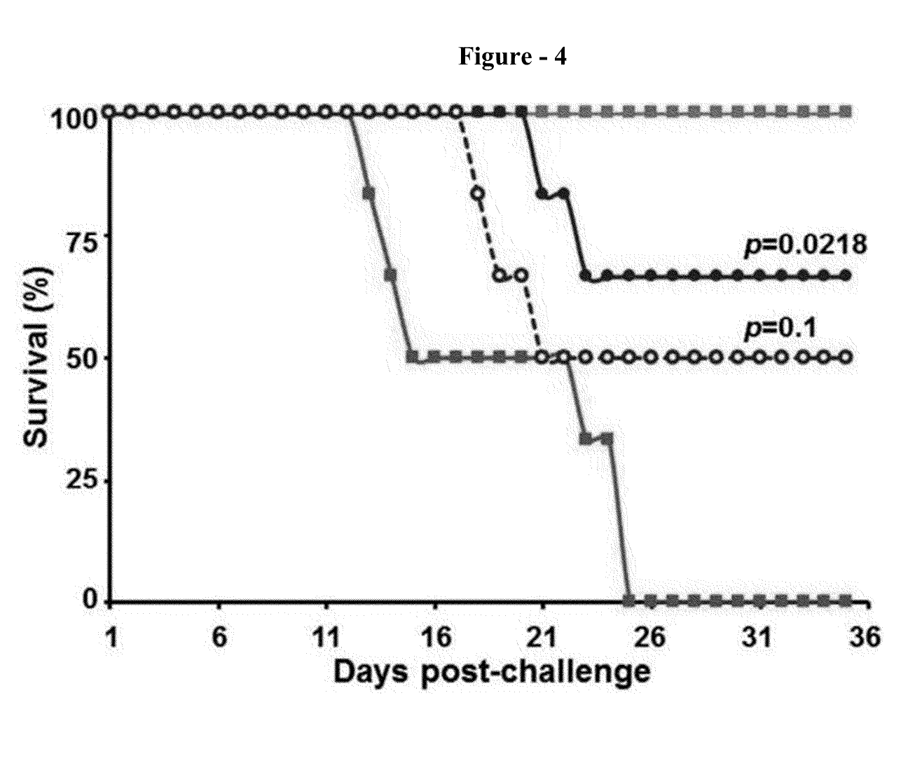

The present inventors have found that the median survival time (MST) of the challenged mice treated orally with the extract (methanol-free) twice a day for 5 days post-challenge increased in a dose-dependent manner. The survival data are present in FIG. 4. The MST of challenged mice was 19 days under the experimental conditions. At a 125 mg/Kg dose, given twice a day for 5 days, survival was 50% and MST was 28 days (p=0.1). This increased to ˜67% when the dosage was doubled. Compared to the placebo-treated (0.25% methyl cellulose) group, the level of protection afforded by 250 mg/Kg dose was statistically significant (p=0.021).

FIG. 4 provides evaluation of the protective efficacy of a methanolic extract in-vivo. AG129 mice (9-12 weeks old) were injected i.p. with 10⁶ PFU brain-passaged DENV-2.

Infected mice were treated orally with 0.25% methyl cellulose (solid red squares) or methanolic extract at 125 mg (empty blue circles) and 250 mg/kg body weight (solid blue circles). Treatment was twice daily for the first 5 days. A control group that was not virus-infected, but which received the higher dose of methanolic extract orally (solid green squares), was tested in parallel. The mice were monitored daily for mortality and the resultant data plotted as Kaplan-Meier survival curves. The p values to assess the statistical significance in the survival rates on day 35 between the methanolic extract-treated and placebo-treated (0.25% methyl cellulose) groups were determined using the Log-rank test.

### (g) Determination of Effect of Paracetamol

Interaction between paracetamol and the extracts of the present invention was assessed in-vitro using type-1 assay format. DENV-3 (˜50 PFUs) and the extracts of the present invention (ranging in concentration from 0 μg/mL to 50 μg/mL) were pre-incubated overnight at 4° C. in a volume of 100 μA, and used to infect LLCMK2 cells in 24-well plates. Parallel infections were set up using pre-incubation mixtures containing paracetamol (1 μg/mL to 100 μg/mL), in addition to DENV and the extracts of the present invention. Mock-infections and DENV only infections (in the absence of the extract and paracetamol) were also set up and analysed in parallel.

The in-vivo effect of the extract of the present invention in the presence and absence of paracetamol was assessed using the Wistar rat pyrexia model. Wistar rats (weighing 180 to 220 g) of either sex were used. The basal temperature of the rats was measured using a digital rectal thermometer; and then the rats were injected subcutaneously (in the intra-scapular region) with 20% brewer's yeast (10 mL/Kg body weight) and allowed to fast overnight with free access to water. At 18 hours post-injection, rectal temperatures were recorded again to identify animals that registered ≧0.7° C. rise in body temperature for inclusion in the study. Groups (n=7 to 9) of febrile rats were orally administered paracetamol (200 mg/Kg), or the extracts of the present invention (200 mg/Kg), or both. Rats in the control group received just the vehicle (0.5% methyl cellulose). This was followed by the recording of the rectal temperature for 3 hours at 30 minute intervals.

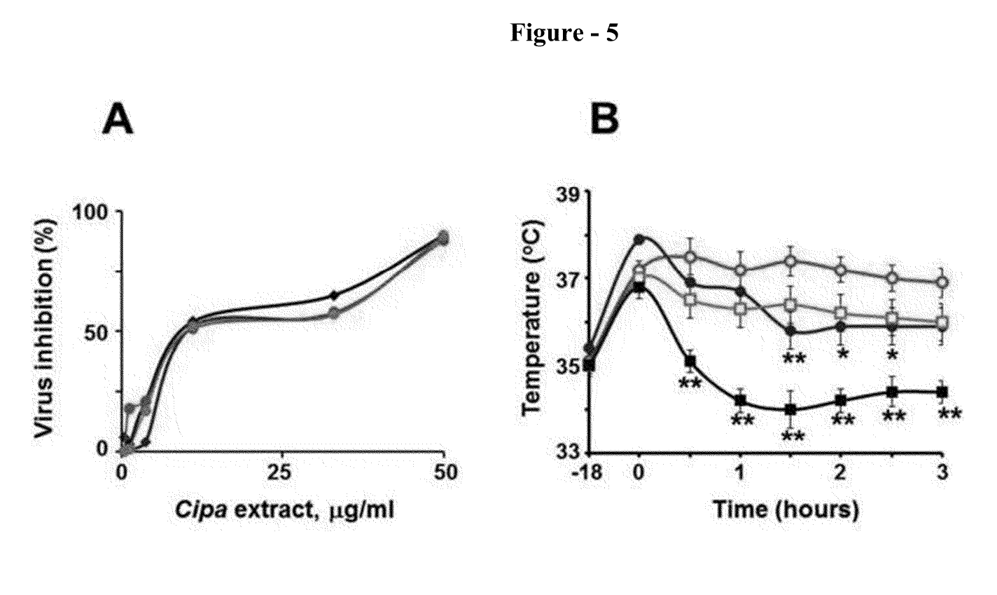

The data from the studies on the methanolic extract and paracetamol are depicted in FIG. 5. A type-1 assay was carried out in which DENV-3 was pre-incubated with serial dilutions of methanolic extract. It was observed that DENV-3 infectivity was inhibited progressively as the methanolic extract concentration increased, with an IC₅₀ value of 6.1 μg/mL. The addition of up to 100 μg/mL paracetamol into the DENV-3/methanolic extract pre-incubation mix did not significantly affect the inhibitory profile of the extract. The calculated IC₅₀ values in the presence of paracetamol at 1, 10, and 100 μg/mL were, respectively, 8.4, 7.4, and 8.5 μg/mL (FIG. 5A). Paracetamol by itself at all concentrations tested did not have any effect on DENV infectivity (plaque counts obtained with DENV-3 alone and DENV-3 plus paracetamol at 100 μg/mL were 43±3 and 45±4, respectively; n=3). The next experiment examined the effect of methanolic extract on the antipyretic activity of paracetamol using the Wistar rat pyrexia model. Surprisingly, this experiment revealed that the methanolic extract possessed intrinsic antipyretic effect (FIG. 5B). When rats, in which fever was induced by subcutaneous injection of brewer's yeast, were treated with methanolic extract, the fever was suppressed at an efficiency that was comparable to that of paracetamol. Surprisingly, co-administration of the methanolic extract with paracetamol had a synergistic effect, resulting in a more pronounced decrease in body temperature.

FIG. 5 provides an analysis of interaction between paracetamol and methanolic extract.

(A) DENV-3 (50 PFU) was pre-incubated with methanolic extract in the absence (solid black diamonds) or presence of 1 μg/mL (solid blue diamonds), 10 μg/mL (solid red circles), or 100 μg/mL (solid green circles) of paracetamol overnight at 4° C., followed by analysis of viral inhibition in a type-1 assay.
(B) Febrile Wistar rats were mock-treated (empty red circles), or treated with paracetamol (solid blue circles), methanolic extract (empty green squares) or a combination of both (solid black squares), followed by monitoring of their rectal temperature for 3 hours post-treatment at regular intervals.

Rectal temperatures between the control and treatment groups were compared using one-way ANOVA followed by Dunnett's multiple comparison test (the single and double stars indicate significant differences in the treatment groups with respect to the control group, corresponding to p values of ≦0.05 and ≦0.01, respectively).

### (h) Determination of Effect on Platelets and Erythrocytes

For ex-vivo studies, erythrocytes were pelleted down in a centrifuge (1500×g, 5 minutes) from freshly collected heparinized human blood, rinsed thoroughly with phosphate buffered saline (PBS, pH 7.4), and used to make a 1% cell suspension in PBS. The extracts of the present invention ranging in concentration from 12.5 mg/L to 400 mg/L were added to the erythrocyte suspension, and then incubated at 37° C. for 1 hour. After this, the samples were spun down, and the absorbance of the supernatant was measured at 576 nm to determine the extent of erythrocyte lysis. Controls, wherein erythrocytes were incubated with buffer alone (0% lysis), DMSO alone, and 0.1% Triton X-100 (100% lysis) were processed in parallel. Basal platelet count in freshly collected heparinized blood and in blood pre-incubated with DMSO (vehicle) or the extracts of the present invention (2 μg/mL to 10 μg/ml) for different durations (1 to 4 hours) was determined using a Beckman Coulter hematology analyser.

For in-vivo studies, four groups (n=5) of Wistar rats were fasted overnight, and then administered orally with the vehicle (0.25% methyl cellulose) or the extracts of the present invention, at three different dosages (100, 300, and 1000 mg/Kg body weight). Blood was collected just before the extract administration (0 hour) and at 1 and 4 hours post-administration. Hematology parameters were measured using ADVIA-120 hematology analyser.

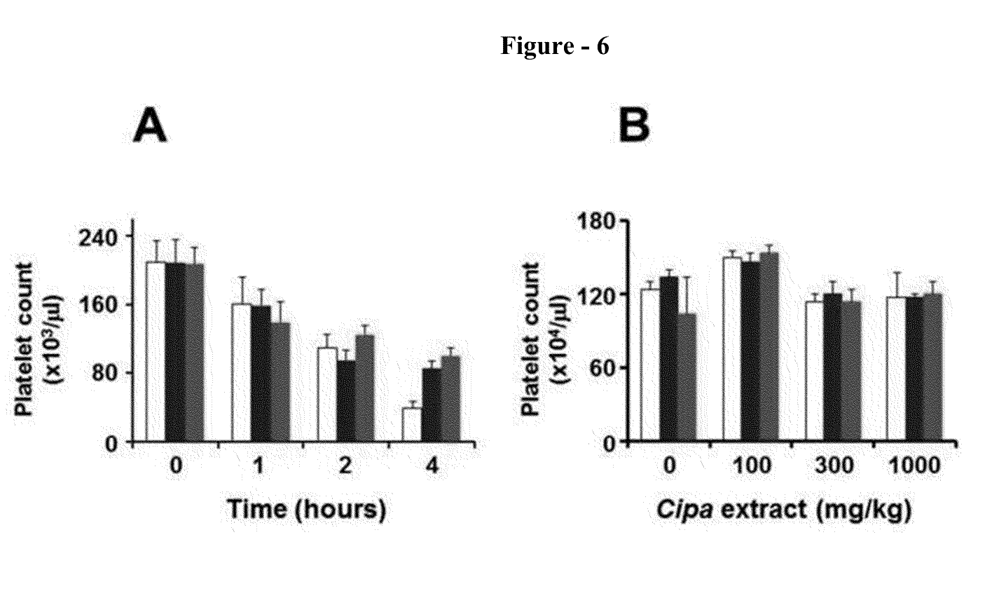

FIG. 6 provides the effect of the methanolic extract on platelets. Whole blood from human volunteers was collected and platelets counts were obtained before and after 1 to 4 hours post-mixing with methanolic extract. The results are shown in FIG. 6A. In the control sample of blood mixed with the vehicle (saline), platelet counts declined steadily over time. Methanolic extract-treated blood samples manifested no statistically significant change in platelet counts with respect to their cognate controls, up to 2 hours (p>0.05). At four hours, the methanolic extract-treated samples displayed significantly higher (p<0.05) platelet counts, compared to the corresponding saline-treated control. Similar results were observed at 2 μg/mL and 10 μg/mL methanolic extract concentrations, indicating that a methanolic extract did not affect platelets adversely. The effect of the methanolic extract on platelets was also evaluated in an in-vivo experiment using Wistar rats. In this experiment, platelet counts were determined in blood drawn from rats which had been given a methanolic extract orally. The results presented in FIG. 6B show that up to 4 hours post treatment, methanolic extract (up to 1000 mg/kg body weight), did not affect platelet counts significantly (p>0.05 at the highest dose of methanolic extract treatment for 4 hours).

FIG. 6 (A): Freshly collected human blood was incubated with saline (white bars) or methanolic extract (at 2 μg/mL: blue bars; at 10 μg/mL: red bars) for up to 4 hours. Aliquots were drawn at the indicated times for determination of platelet counts.

FIG. 6 (B): Wistar rats were orally administered 0.25% methyl cellulose containing methanolic extract ranging from 0 to 1000 mg/Kg body weight.

Fresh blood collected from these rats at 0 (white bars), 1 (blue bars), and 4 (red bars) hours post-administration, was analysed for platelet counts. For both panels, data shown are mean values (n=5); the vertical bars represent standard deviation (SD).

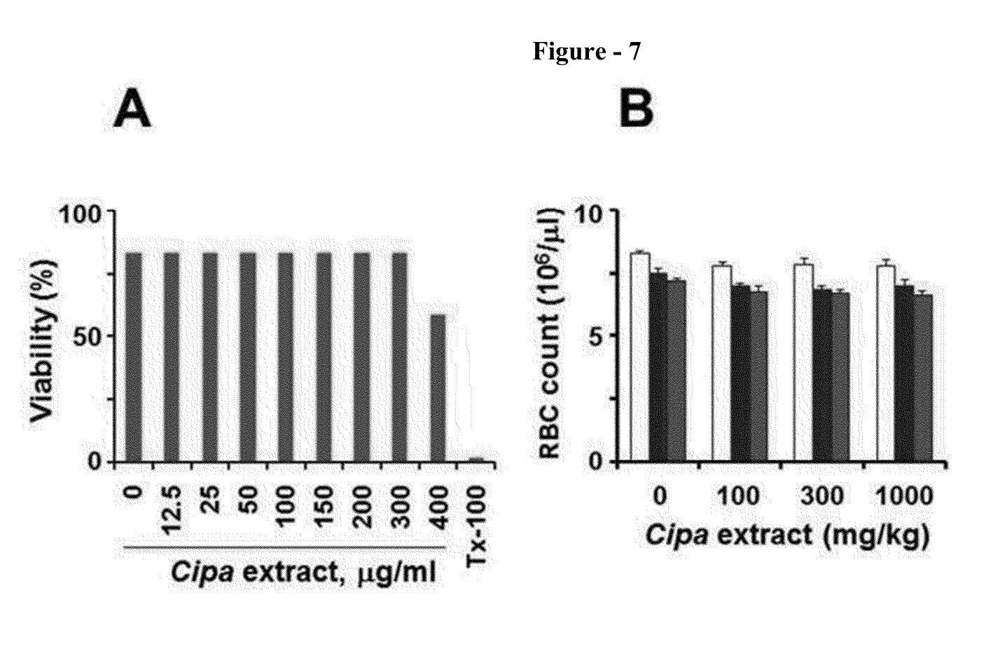

The effect of methanolic extract on erythrocytes was also assessed, both in ex-vivo and in-vivo assays, as done for the platelets above. FIG. 7 provides effect of methanolic extract on RBCs. Incubation of freshly collected human erythrocytes with methanolic extract at concentrations up to 400 μg/L did not cause discernible haemolysis (FIG. 7A). The blood samples withdrawn from the Wistar rats (given methanolic extract, described above) were also analysed for erythrocyte cell counts. This analysis once again revealed that methanolic extract (at concentrations as high as 1000 mg/Kg body weight) did not affect erythrocyte counts in the blood of Wistar rats up to 4 hours post-administration (FIG. 7B). The difference in erythrocyte counts between the treated and untreated rats was not statistically significant (p>0.05). The inventors also analyzed total leucocyte and differential counts in the blood of methanolic extract-treated Wistar rats (described in FIGS. 6B and 7B) and no significant difference was found.

FIG. 7 (A): Erythrocytes from freshly collected human blood were incubated for 1 hour at 37° C., with varying concentrations of methanolic extract (0 μg/mL to 400 μg/ml), followed by measurement of haemolysis at 576 nm. TX-100 represents a control in which an equivalent aliquot of erythrocytes were treated with Triton X-100 to achieve complete lysis.

FIG. 7 (B): Fresh blood collected from the methanolic extract-treated Wistar rats at (0 white bars), 1 (blue bars), and 4 (red bars) hours post-administration was analysed for RBC counts. Data shown are mean values (n=5); the vertical bars represent SD.

### (i) Cytokine Release Assay

Freshly collected heparinized blood was diluted with an equal amount of RPMI 1640 medium, then layered on Ficoll Hypaque 1077, and then centrifuged at 2,500 rpm for 25 minutes at room temperature. The upper layer was discarded and the fluffy layer at the interphase was harvested, then rinsed, and then re-suspended in RPMI 1640 at 5×10⁵ cells/mL to obtain human peripheral blood mononuclear cells (PBMCs). Freshly collected PBMCs were seeded in 96-well plates (10⁵ cells/well) and treated with the extracts of the present invention at different dilutions (in RPMI 1640), followed by 30 minutes of incubation at room temperature on a rotary shaker (200 rpm). Next, the wells were treated with 50 μl (4 μg/mL) lipopolysaccharide and allowed to incubate for a further 30 minutes at room temperature. The volume per well was made up to 200 μl using RPMI+10% FCS, and the plates were incubated overnight at 37° C. in a CO₂ incubator. Negative controls (no lipopolysaccharide treatment) were run in parallel. The plates were centrifuged (3000 rpm, 10 minutes) to obtain clarified supernatants for TNF-α and IL-1β determinations using commercial ELISA kits.

Methanolic extract efficiently suppressed the secretion of TNF-α and IL-1β with IC₅₀ values of 6.1±1.3 and 5.7±2.7 μg/mL, respectively. An MTT assay showed that at these concentrations, methanolic extract has no discernible cytotoxicity in both cell lines tested (CC₅₀=78.9 μg/ml in HepG2; >200 μg/ml in LLCMK2). These data show the anti-inflammatory activity of the methanolic extract.

### (j) Toxicology

Groups of 5 adult Wistar rats were orally administered 4 mL 0.25% methyl cellulose (vehicle)/Kg or 4 mL vehicle containing 400 mg to 2000 mg of the extracts of the present invention/kg, once daily for 7 days (in accordance with OECD guidelines—407). During this period, food intake, body weight, and clinical signs were monitored daily. At the end of the experiment, animals were euthanized, followed by the determination of hematological (Hb, WBC count, RBC count, platelet count, and hematocrit) and biochemical (SGOT, SGPT, total protein, serum albumin, total cholesterol, urea, creatinine, and random sugar) parameters. Necropsy was performed. Organ weights were recorded and histopathology was done.

The results showed that animals treated with up to 2000 mg/kg body weight did not manifest any significant changes in any of these parameters compared to vehicle treated controls.

We claim:

1. A pharmaceutical composition for use in the treatment of dengue virus infection in mammals, comprising:
(a) an extract of Cissampelos pareira; and
(b) paracetamol
wherein (a) and (b) are administered together as a single pharmaceutical composition or are co-administered simultaneously or sequentially.

2. A method of treating dengue virus infection comprising administering the pharmaceutical composition of claim 1.

3. A method of reducing the viral load at an early stage in the treatment of dengue virus infection in mammals comprising administering an extract of Cissampelos pareira.

4. A method of treating dengue virus infection in mammals comprising administering an extract of Cissampelos pareira, wherein the extract exhibits a platelet protective effect.

5. A method of treating dengue infection in mammals comprising administering an extract of Cissampelos pareira, wherein the extract exhibits an erythrocyte protective effect.

6. The method of any of claims 1-5, wherein the extract is selected from an alcoholic extract, a hydroalcoholic extract, or an aqueous extract.

7. The method of claim 6, wherein the alcoholic extract is selected from the group consisting of methanolic, ethanolic, n-propanolic, isopropanolic, n-butanolic, iso-butanolic, and t-butanolic extracts.

8. A method for reducing viral load at an early stage in the treatment of dengue virus infection in mammals comprising administering an extract of Cissampelos pareira, wherein the extract exhibits a platelet protective effect.

9. A method for reducing viral load at an early stage in the treatment of dengue virus infection in mammals, comprising administering an extract of Cissampelos pareira, wherein the extract exhibits an erythrocyte protective effect.

10. A pharmaceutical composition comprising the extract of claim 8 or claim 9 and one or more pharmaceutically acceptable excipients.

11. The pharmaceutical composition of claim 10, wherein the pharmaceutically acceptable excipients are selected from the group comprising diluents, binders, disintegrants, lubricants, glidants, polymers, flavoring agents, surfactants, preservatives, antioxidants, buffers, and tonicity modifying agents.

12. The method of claim 8 or claim 9, wherein the extract is selected from an alcoholic extract, a hydroalcoholic extract, or an aqueous extract.

13. The method of claim 12, wherein the alcoholic extract is selected from the group consisting of methanolic, ethanolic, n-propanolic, isopropanolic, n-butanolic, iso-butanolic, and t-butanolic extracts.
